(19) United States
(12) Patent Application Publication
(10) Pub. No.: US 2007/0082033 A1
(43) Pub. Date: Apr. 12, 2007

(54) Substrate containing lipids

(75) Inventors: Albrecht Doerschner, Hamburg (DE); Magalie Racine, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/578,727

(22) PCT Filed: Nov. 4, 2004
(86) PCT No.: PCT/EP04/52795

§ 371 Date: Sep. 28, 2006

(30) Foreign Application Priority Data
Nov. 10, 2003 (DE) 103 52 370.7

(51) Int. Cl. A61K 9/70

(52) U.S. Cl. 424/443

(57) ABSTRACT

Cleansing article of a textile that is impregnated with a preparation containing a) one or more anionic surfactants in a total concentration of from 1 to 30% ,by weight and b) one or more lipids liquid or viscous at room temperature in a total concentration of at least 8% by weight, respectively based on the total weight of the preparation, its production and use.

The present invention relates to a cleansing article, its production and use.

Cleansing the human body means the removal of (environmental) dirt and thus causes an increase in psychological and physical well being. Cleaning the surface of the skin and hair is a very complex process dependent on many parameters. On the one hand, substances from outside, such as, e.g., hydrocarbons or inorganic pigments from various environments and residue from cosmetics or also undesirable microorganisms are to be removed as completely as possible. On the other hand, endogenous secretions, such as sweat, sebum, cutaneous scales and dandruff can be washed away without serious interference in the physiological balance of the skin.

Solid cleansing substrates or textiles, in particular wipes, represent a special product form for cleansing preparations. These can be already impregnated with the cleansing preparation by the manufacturer (a combination for which within the scope of the present invention, the term “cleansing article” is also used) and thus have the advantage that the preparation is already provided therein in the correct dosage. Furthermore, they avoid the disadvantage of preparations stored in bottles, the packaging of which can break and the contents of which can leak out. The further advantages of cleansing substrates/textiles also include the facts that they can easily be taken along in counted quantities when traveling and as a rule no more water is necessary for their application.

Cleansing substrates/textiles are made from textiles. Textiles may be woven, knitted or machine-knitted, or be present as composite material (nonwoven textile). In most cases, composite materials are used (for cost reasons). In the case of composite materials, the fabric is not produced by warp and weft or stitch formation, but by interlocking, and/or cohesive and/or adhesive bonding of textile fibers. According to DIN 61210 T2, composite materials may be distinguished by nonwoven fabrics, paper, batting, and felt. Nonwoven fabrics are loose materials produced from spun fibers (i.e., fibers of a limited length), or filaments (endless fibers), in most cases made of polypropylene, polyester, or viscose, the cohesion of which is generally provided by the fibers intrinsically holding together. In this regard, the individual fibers may have a preferred orientation (oriented or cross-laid nonwoven fabrics), or be unoriented (entangled nonwoven fabrics). The nonwoven fabrics may be mechanically bonded by needle punching, stitching, or entangling by means of strong water jets. Adhesively bonded nonwoven fabrics are produced by gluing the fibers together with liquid binding agents (for example, acrylate polymers, SBR/NBR, polyvinyl ester, polyurethane dispersions), or by melting or dissolving so-called binder fibers that are added to the nonwoven fabric during its production. In the case of cohesive bonding, the fiber surfaces are partially dissolved by suitable chemicals and bonded by pressure or fused at an increased temperature [J. Falbe, M. Regnitz: Römpp-Chemie-Lexikon, 9^{th} edition, Thieme-Verlag, Stuttgart (1992)].

Substrates that are impregnated with cosmetic preparations and in particular wipes may be produced in different ways: in a so-called “dip method”, the wipe is immersed into a dip bath or pulled through a bath. This method is especially suited for paper wipes and less suited for nonwoven fabrics, since the latter absorb too much liquid (=preparation), and when being repackaged, subsequently are found in puddles of the preparation that has been released again.

A second variant is the “spraying method,” wherein the preparation is sprayed onto the advancing fabric. While this method is suitable for all textiles, it does not permit applying heavily foaming preparations to the wipe, since the foam development becomes too great in the spraying method.

As further methods, so-called squeegee methods are used. Here nonwoven or fabric webs advance along doctor blades, doctor bars, or doctor nozzles, which continuously receive an impregnation solution. Different degrees of impregnation may be adjusted, among other things, by varying the contact pressure and the drawing rate of the fabric.

However, substrates/wipes that can be produced according to the prior art, which are impregnated with cleansing preparations or other cosmetic preparations (=cleansing articles) have a series of disadvantages. In particular it has proven difficult to find the correct balance of cleansing power and refatting:
The wipes with “low-lipid,” aqueous preparations have a high foaming power and a high cleansing power. However, with such wipes too large an amount of lipids is removed during the cleansing of the skin, the skin is dried out and damaged.
Wipes with “low lipid,” aqueous preparations usually foam upon impregnation so much that they cannot be produced by means of “spray methods.” Also with the other conventional production methods (squeegee method, dip bath) the good foaming power of the preparation makes the production process extremely susceptible to faults.
Wipes with heavily refatting preparations (e.g., emulsions, “nonaqueous” lipid impregnations), on the other hand, have an at most slight cleansing power. This is due not least to the low foaming power of the preparations, because as a rule lipids have a defoaming effect. A small amount of foam is to be equated with a small surface at which a mass transfer (=cleansing power) of skin surface to the cleansing preparation can take place.

It was therefore the object of the present invention to eliminate the disadvantages of the prior art and to develop a cleansing article as well as a production method for a cleansing article which has a high cleansing power and a high refatting capability with respect to the skin.

Surprisingly, the objects are attained by a cleansing article of a textile that is impregnated with a preparation containing
a) one or more anionic surfactants in a total concentration of from 1 to 30% by weight and
b) one or more lipids which are liquid or viscous at room temperature in a total concentration of at least 8% by weight, each based on the total weight of the preparation.

The object is further attained through a method for producing a cleansing article, characterized in that a textile is impregnated with a preparation containing
a) one or more anionic surfactants in a total concentration of from 1 to 30% by weight and
b) one or more lipids which are liquid or viscous at room temperature in a total concentration of at least 8% by weight,
   each based on the total weight of the preparation, by immersing the textile in a dip bath, spraying the textile with the preparation or by wiping doctor blades, doctor bars, or doctor nozzles of the textile on doctor blades, doctor bars, or doctor nozzles discharging the preparation, and by the use of a preparation containing
a) one or more anionic surfactants in a total concentration of from 1 to 30% by weight and
b) one or more lipids which are liquid or viscous at room temperature in a total concentration of at least 8% by weight, each based on the total weight of the preparation, for impregnating cosmetic cleansing wipes that during application to the skin foam and refat the skin with lipids.

The cleansing articles according to the invention (within the scope of this disclosure these also always include the cleansing articles produced according to the production method according to the invention and the cleansing articles which are present upon the use according to the invention of the preparation according to the invention) during their application have a high refatting effect with respect to the skin. Furthermore, during application (“rubbing onto the skin”) they develop a pleasant finely bubbled foam. The foaming occurring during production is thereby surprisingly slight.

A number of cleansing wipes are known from the prior art. Thus, WO 03/005982 and WO 03/005983 describe lipid-impregnated wipes, which, however, do not contain any surfactants in the preparations. WO 97/06306 describes wipes with an emulsion-based impregnation. U.S. Pat. No. 3,795,624 describes wipes that are impregnated with a lipid containing cleansing preparation on the basis of non-ionic surfactants. EP 0934056 and WO 98/18442 describe preparations with a content of oil components of up to 6% by weight of the preparation. WO 99 55303 describes a two-stage production method for impregnated wipes, in which the active component of the preparation is applied to the wipe after the surfactant preparation applied beforehand has dried. However, these documents cannot show the way to the present invention.

According to the invention, the cleansing articles according to the invention preferably contain
a) one or more anionic surfactants in a total concentration of from 2 to 25% by weight and
b) one or more lipids which are liquid or viscous at room temperature in a total concentration of at least 8% by weight,
   each based on the total weight of the preparation.

It is particularly preferred according to the invention if the preparation according to the invention contains one or more lipids liquid or viscous at room temperature in a total concentration of at least 20% by weight, based on the total weight of the preparation.

Lipids which are liquid or viscous at room temperature are understood according to the invention to be those that have a viscosity of less than 10,000 mpas. The viscosities are determined according to the invention with the aid of a viscosimeter of the Viskotester VT 02 type by Haake (temperature: 25° C., spindle diameter 24 mm, rotor speed 62.5 1/min).

The textiles according to the invention can be smooth or surface-structured. Surface-structured substrates are preferred according to the invention.

With the textiles according to the invention, the fabric can be formed by warp and weft, by mesh formation or by intertwining, and/or cohesive and/or adhesive bonding of textile fibers. In this connection, it is preferred according to the invention if the substrate is a composite.

According to the invention, preference is given to using textiles in the form of nonwoven fabrics, in particular of water-jet-consolidated and/or water-jet-impressed nonwoven fabric. The substrates may also advantageously be implemented in the form of a wad, perforated nonwoven fabric or net.

Textiles in the form of wipes are particularly preferred according to the invention.

Textiles according to the invention can have macroimpressions in any desired pattern. The choice to be made depends firstly on the impregnation to be applied and secondly on the field of use in which the subsequent textile is to be used.

It has proven advantageous for the textile to have a weight of from 20 to 120 g/m², preferably 30 to 80 g/m², particularly preferably 40 to 60 g/m² (measured at 20° C.±2° C. and at a room air humidity of 65%±5% for 24 hours).

The thickness of the textile is preferably 0.2 mm to 2 mm, in particular 0.4 mm to 1.5 mm, very particularly preferably 0.6 mm to 0.9 mm.

Starting materials for the nonwoven fabric of the wipe which can be used are generally all organic and inorganic natural and synthetic-based fibrous materials. Examples which may be given are viscose, cotton, cellulose, jute, hemp, sisal, silk, wool, polypropylene, polyester, polyethylene terephthalate (PET), aramide, nylon, polyvinyl derivatives, polyurethanes, polylactide, polyhydroxyalkanoate, cellulose esters and/or polyethylene, and also mineral fibers, such as glass fibers or carbon fibers. However, the present invention is not limited to the materials specified, it being possible instead to use a large number of further fibers for forming the nonwoven fabric. For the purposes of the present invention, it is particularly advantageous if the fibers used are not water-soluble.

In an advantageous embodiment of the nonwoven fabric, the fibers comprise a mixture of 60% to 80% viscose with 40% to 20% PET, in particular 70% viscose and 30% PET. A mixture of 70% viscose and 30% PET is particularly advantageous.

According to the invention, a textile according to the invention can advantageously have a mixture of three different fibrous materials. In such a case, a mixture of 10% to 80% viscose with 20% to 90% polyester and 0 to 30% cotton is preferred. According to the invention, particular preference is given to a mixture of 40% viscose and 50% PET and 10% cotton.

Also particularly advantageous are fibers of high-strength polymers, such as polyamide, polyester and/or highly drawn polyethylene.

Moreover, the fibers can also be dyed in order to be able to emphasize and/or enhance the visual attractiveness of the nonwoven fabric. The fibers can additionally comprise UV stabilizers and/or preservatives.

The fibers used to form the textile preferably have a water-absorption rate of more than 60 mm/[10 min] (measured using the EDANA test 10.1-72), in particular more than 80 mm/[10 min].

Furthermore, the fibers used to form the textile preferably have a water-absorption capacity of more than 5 g/g (measured using the EDANA test 10.1-72), in particular more than 8 g/g.

Advantageous textiles for the purposes of the present invention have a tear strength of, in particular,

| | | [N/50 mm] |
|---|---|---|
| in the dry state | machine direction | >60, preferably >80 |
| | transverse direction | >20, preferably >30 |
| in the impregnated state | machine direction | >4, preferably >60 |
| | transverse direction | >10 preferably >20 |

The expandability of advantageous textiles is preferably

| | | |
|---|---|---|
| in the dry state | machine direction | 15% to 100%, preferably |
| | | 20% and 50% |
| | transverse direction | 40% to 120%, preferably |
| | | 50% and 85% |
| in the impregnated state | machine direction | 15% to 100%, preferably |
| | | 20% and 40% |
| | transverse direction | 40% to 120%, preferably |
| | | 50% and 85% |

Advantageously according to the invention also so-called crepe papers or tissues can be used as textiles. Crepe papers (e.g., toilet papers are paper types made expandable and supple through wet or dry creping; tissue is a particularly thin, soft, mainly wood-free material with fine (dry) creping, which is composed of one or more layers and is very absorbent. The base weight of the individual layer is generally less than 25 g/m² before creping. In addition to the fibrous materials, the papers can also contain further substances, so-called paper additive products. These include fillers (e.g., kaolin, chalk, titanium dioxide) to improve smoothness and printability and surface quality, colorants and pigments for dyeing or surface coloring, binding agents (e.g., starch, casein and other proteins, synthetic dispersions, resin sizes and the like) for strengthening the fiber structure and for binding fillers and pigments and for increasing the water resistance, optical brighteners to increase the whiteness, retention agents (e.g., aluminum sulfate and synthetic cationic substances) to retain the fine materials and fillers during production, “de-inking chemicals” for treating recovered paper and various other substances, such as, e.g., wetting agents, antifoaming agents, preservatives, slime control agents, softeners, antiblocking agents, antistatic agents, hydrophobizing agents, etc.

It is advantageous for the purposes of the present invention if the impregnating medium has a viscosity of 100 to 2000 mpas, preferably 200 to 1000 mpas. The viscosities are determined according to the invention with the aid of a viscosimeter of the Viskotester VT 02 type by Haake (temperature: 25° C., spindle diameter 24 mm, rotor speed 62.5 1/min.).

According to the invention, the degree of impregnation of the textile (i.e., the weight ratio of impregnation preparation to textile) of the cleansing article according to the invention is advantageously from 100% to 1000%, and preferably 200% to 500%.

The cleansing articles according to the invention are characterized in that in the impregnation preparation a) one or more compounds are selected from the list of the following compounds as anionic surfactants: sodium myreth sulfate, sodium laureth sulfate, monoisopropanolamine laureth sulfate, sodium acyl glutamate sodium lauroyl sarcosinate, sodium methyl cocoyl taurate, dioctyl sodium sulfosuccinate.

Moreover, the preparations can contain other anionic, cationic, amphoteric and non-ionic surfactants. These can be selected, e.g., from the list of the following compounds:

A. Anionic Surfactants

Anionic surfactants advantageously additionally to be used are

Acylamino acids (and salts thereof, such as
1. Acyl glutamates, such as di-TEA palmitoyl aspartate and sodium caprylic/capric glutamate,
2. Acyl peptides, for example, palmitoyl hydrolysed milk protein, sodium cocoyl hydrolysed soybean protein and sodium/potassium cocoyl hydrolysed collagen,
3. Sarcosinates, for example, myristoyl sarcosine, TEA-lauroyl sarcosinate, and sodium cocoyl sarcosinate,
4. Taurates, for example, sodium lauroyl taurate
5. Acyl lactylates, lauroyl lactylate, caproyl lactylate
6. Alaninates

Carboxylic acids and derivatives, such as
1. Carboxylic acids, for example, lauric acid, aluminum stearate, magnesium alkanolate and zinc undecylenate,
2. Ester carboxylic acids, for example, calcium stearoyl lactylate, laureth-6 citrate and sodium PEG-4 lauramide carboxylate,
3. Ether carboxylic acids, for example, sodium laureth-13 carboxylate and sodium PEG-6 cocamide carboxylate,

Phosphoric esters and salts, such as, for example, DEA oleth-10 phosphate and dilaureth-4 phosphate,

Sulfonic acids and salts, such as
1. Acyl isethionates, e.g., sodium/ammonium cocoyl isethionate
2. Alkylarylsulfonates,
3. Alkylsulfonates, for example, sodium cocomonoglyceride sulfate, sodium C₁₂₋₁₄-olefinsulfonate, sodium lauryl sulfoacetate and magnesium PEG-3 cocamide sulfate,
4. Sulfosuccinates, for example, disodium laureth sulfosuccinate, disodium lauryl sulfosuccinate, disodium undecylenamido-MEA sulfosuccinate and PEG-5 lauryl citrate sulfosuccinate and

Sulfuric esters, such as
1. Alkyl ether sulfate, for example, ammonium, magnesium, TIPA laureth sulfate, sodium myreth sulfate and sodium C₁₂₋₁₃ pareth sulfate,
2. Alkyl sulfates, for example, sodium, ammonium and TEA lauryl sulfate.

B. Cationic Surfactants

Cationic surfactants which can be used advantageously in addition are
1. Alkylamines,
2. Alkylimidazoles,
3. Ethoxylated amines and
4. Quaternary surfactants,
5. Ester quats

Quaternary surfactants contain at least one N atom which is covalently bonded to 4 alkyl and/or aryl groups. Irrespective of the pH, this leads to a positive charge. Alkylbetaine, alkylamidopropylbetaine and alkyl amidopropylhydroxysulfaine are advantageous quaternary surfactants. For the purposes of the present invention, cationic surfactants may also preferably be chosen from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example, lauryl- or cetylpyridinium chloride, imidazoline derivatives and compounds with cationic character, such as amine oxides, for example, alkyldimethylamine oxides or alkylaminoethyldimethylamine oxides. In particular, the use of cetyltrimethylammonium salts is advantageous.

C. Amphoteric Surfactants

Amphoteric surfactants which can be used advantageously in addition are
1. Acyl/dialkylethylenediamine, for example, sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,
2. N-alkylamino acids, for example, aminopropylalkylglutamide, alkylaminopropionic acid, sodium alkylimidodipropionate and lauroamphocarboxyglycinate.

D. Nonionic Surfactants

Nonionic surfactants which can be used advantageously in addition are
1. Alcohols,
2. Alkanolamides, such as cocamides MEA/DEA/MIPA,
3. Amine oxides, such as cocoamidopropylamine oxide,
4. Esters that are formed by esterification of carboxylic acids with ethylene oxide, glycerol, sorbitan or other alcohols,
5. Ethers, for example, ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside.
6. Sucrose esters, sucrose ethers
7. Polyglycerol esters, diglycerol esters, monoglycerol esters
8. Methyl glucose esters, esters of hydroxy acids.

It is also advantageous to use a combination of anionic and/or amphoteric surfactants with one or more nonionic surfactants.

The cleansing articles according to the invention are characterized in that in the impregnation preparation, one or more compounds selected from the group mineral oil, C₁₂₋₁₅ alkyl benzoate, octyldodecanol, soy bean oil, cetyl palmitate, are used as lipids b) which are liquid or viscous at room temperature.

Moreover, the impregnation preparation of the cleansing articles according to the invention can advantageously contain further lipophilic components. These can be selected, e.g., from the list of the following compounds:

Polar Oils

Polar oils are, e.g., those from the group of lecithins and fatty acid triglycerides, namely the triglycerol esters of saturated and/or unsaturated, branched and/or unbranched alkanecarboxylic acids having a chain length of from 8 to 24, in particular 12 to 18 C atoms. The fatty acid triglycerides can advantageously be chosen, for example, from the group of synthetic, semi-synthetic and naturally occurring oils, for example, olive oil, sunflower oil, soybean oil, peanut oil, rapeseed oil, almond oil, palm oil, coconut oil, castor oil, wheat germ oil, rape-seed oil, grape-seed oil, safflower oil, evening primrose oil, macadamia nut oil and many others.

Particularly advantageous polar lipids for the purposes of the present invention are all native lipids, such as, e.g., olive oil, sunflower oil, soybean oil, peanut oil, rapeseed oil, almond oil, palm oil, coconut oil, castor oil, wheat germ oil, grape-seed oil, safflower oil, evening primrose oil, macadamia nut oil, corn oil, avocado oil and the like and those listed below:

| | | | Polarity |
|---|---|---|---|
| Manufacturer | Trade Name | INCI Name | [mN/m] |
| Condea Chemie | Isofol 14 T | Butyl decanol (+) hexyl octanol | 19.8 |
| | | (+) hexyl decanol (+) butyl | |
| | | octanol | |
| Lipochemicals Inc./ | Lipovol MOS-130 | Tridecyl stearate(+) tridecyl | 19.4 |
| USA | | trimellitate(+) dipentaerythrityl | |
| | | hexacaprylate/hexacaprate | |
| (Induchem) | | | |
| | Castor oil | | 19.2 |
| CODEA Chemie | Isofol ester 0604 | | 19.1 |
| Huels | Miglyol 840 | Propylene glycol | 18.7 |
| CONDEA Chemie | | dicaprylate/dicaprate | |
| CONDEA Chemie | Isofol 12 | Butyl octanol | 17.4 |
| Goldschmidt | Tegosoft SH | Stearyl heptanoate | 17.8 |
| | Avocado oil | | 14.5 |
| Henkel Cognis | Cetiol B | Dibutyl adipate | 14.3 |
| ALZO (ROVI) | Dermol 488 | PEG 2 | 10.1 |
| | | Diethylenehexanoate | |
| Condea Augusta | Cosmacol ELI | C12-13 alkyl lactate | 8.8 |
| S.P.A. | | | |
| ALZO (ROVI) | Dermol 489 | Diethylene glycol dioctanoate/ | 8.6 |
| | | diisononanoate | |
| Condea Augusta | Cosmacol ETI | Di-C12/13 alkyl tartrate | 7.1 |
| S.P.A. | | | |
| Henkel Cognis | Emerest 2384 | Propylene glycol monoisostearate | 6.2 |
| Henkel Cognis | Myritol 331 | Cocoglycerides | 5.1 |
| Unichema | Prisorine 2041 GTIS | Triisostearin | 2.4 |

Furthermore, the oil phase can advantageously be chosen from the group of the dialkyl ethers, the group of the saturated or unsaturated, branched or unbranched alcohols. It is particularly advantageous if the oil phase has a content of C₁₂₋₁₅ alkyl benzoate or consists entirely thereof.

In addition, the oil phase can advantageously be chosen from the group of Guerbet alcohols. Guerbet alcohols are named after Marcel Guerbet who described their preparation for the first time. They are formed according to the equation 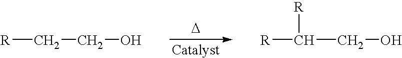
by oxidation of an alcohol to an aldehyde, by aldol condensation of the aldehyde, elimination of water from the aldol and hydrogenation of the allyl aldehyde. Guerbet alcohols are liquid even at low temperatures and effect virtually no skin irritations. They can be used advantageously as fatting, superfatting and also refatting constituents in skin care and hair care compositions.

The use of Guerbet alcohols in cosmetics is known per se. Such species are then in most cases characterized by the structure

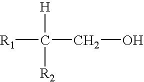

Here, R₁ and R₂ are usually unbranched alkyl radicals.

According to the invention, the Guerbet alcohol(s) is/are advantageously chosen from the group in which
R₁=propyl, butyl, pentyl, hexyl, heptyl or octyl and
R₂=hexyl, heptyl, octyl, nonyl, decyl, undecyl, dodecyl, tridecyl or tetradecyl.

Guerbet alcohols preferred according to the invention are 2-butyloctanol it has the chemical structure 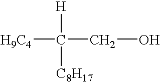
and is available, for example, under the trade name Isofol® 12 from Condea Chemie GmbH- and 2-hexyldecanol—it has the chemical structure 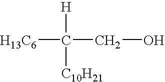
and is available, for example, under the trade name Isofol® 16 from Condea Chemie GmbH. Mixtures of Guerbet alcohols according to the invention can also be used advantageously according to the invention. Mixtures of 2-butyloctanol and 2-hexyldecanol are available, for example, under the trade name Isofol® 14 from Condea Chemie GmbH.

The total amount of Guerbet alcohols in the finished cosmetic or dermatological preparations is advantageously chosen from the range up to 25.0% by weight, preferably 0.5 to 15.0% by weight, based on the total weight of the preparations.

Any mixtures of such oil and wax components can also be used advantageously for the purposes of the present invention. It may optionally also be advantageous to use waxes, for example cetyl palmitate, as the sole lipid component of the oil phase.

Particularly advantageous medium-polar lipids for the purposes of the present invention are the substances listed below:

| | | | Polarity |
|---|---|---|---|
| Manufacturer | Trade Name | INCI Name | [mN/m] |
| Stearinerie Dubois | DUB VCI 10 | Isodecyl neopentanoate | 29.9 |
| Fils | | | |
| ALZO (ROVI) | Dermol IHD | Isohexyldecanoate | 29.7 |
| ALZO (ROVI) | Dermol 108 | Isodecyl octanoate | 29.6 |
| | Dihexyl ether | Dihexyl ether | 29.2 |
| ALZO (ROVI) | Dermol 109 | Isodecyl 3,5,5 trimethyl hexanoate | 29.1 |
| Henkel Cognis | Cetiol SN | Cetearyl isononanoate | 28.6 |
| Unichema | Isopropylpalmitat | Isopropyl palmitate | 28.8 |
| Dow Corning | DC Fluid 345 | Cyclomethicone | 28.5 |
| Dow Corning | Dow Corning | Cyclopolydimethylsiloxane | 28.5 |
| | Fluid 244 | | |
| Nikko Chemicals | Jojobaöl Gold | | 26.2 |
| Superior Jojoba Oil | | | |
| Gold | | | |
| Wacker | Wacker AK 100 | Dimethicone | 26.9 |
| ALZO (ROVI) | Dermol 98 | 2-Ethylhexanoic acid 3,5,5 | 26.2 |
| | | trimethyl ester | |
| Dow Corning | Dow Corning | Open | 25.3 |
| | Fluid 246 | | |
| Henkel Cognis | Eutanol G | Octyl dodecanol | 24.8 |
| Condea Chemie | Isofol 16 | Hexyl decanol | 24.3 |
| ALZO (ROVI) | Dermol 139 | Isotridecyl 3,5,5 | 24.5 |
| | | trimethyl hexanonanoate | |
| Henkel Cognis | Cetiol PGL | Hexyldecanol (+) hexyl decyl | 24.3 |
| | | laurate | |
| | Cegesoft C24 | Octyl palmitate | 23.1 |
| Gattefossé | M.O.D. | Octyl dodeceyl myristate | 22.1 |
| | Macadamia nut | | 22.1 |
| | oil | | |
| Bayer AG, | Silikonöl VP 1120 | Phenyl trimethicone | 22.7 |
| Dow Corning | | | |
| CONDEA Chemie | Isocarb 12 | Butyl octanoic acid | 22.1 |
| Henkel Cognis | Isopropylstearat | Isopropyl stearate | 21.9 |
| WITCO, | Finsolv TN | C12-15 alkyl benzoate | 21.8 |
| Goldschmidt | | | |
| Dr. Straetmans | Dermofeel BGC | Butylene glycol caprylate/ | 21.5 |
| | | caprate | |
| Unichema | Miglyol 812 | Caprylic/capric triglyceride | 21.3 |
| Huels | | | |
| Trivent (via S. Black) | Trivent OCG | Tricaprylin | 20.2 |
| ALZO (ROVI) | Dermol 866 | PEG diethylhexanoate/ | 20.1 |
| | | diisononanoate/ethylhexyl | |
| | | isononanoate | |

Nonpolar Oils

Nonpolar oils are, for example, those which are chosen from the group of branched and unbranched hydrocarbons and hydrocarbon waxes, in particular Vaseline (petrolatum), paraffin oil, squalane and squalene, polyolefins, hydrogenated polyisobutenes. Among the polyolefins, polydecenes are the preferred substances.

Particularly advantageous nonpolar lipids for the purposes of the present invention are the substances listed below:

| | | | Polarity |
|---|---|---|---|
| Manufacturer | Trade Name | INCI Name | [mN/m] |
| Total SA | Ecolane 130 | Cycloparaffin | 49.1 |
| Neste PAO N.V. | Nexbase 2006 FG | Polydecene | 46.7 |
| (Supplier Hansen | | | |
| & Rosenthal) | | | |
| Chemische Fabrik | Polysynlane | Hydrogenated polyisobutene | 44.7 |
| Lehrte | | | |
| Wacker | Wacker Silikonöl AK 50 | Polydimethylsiloxane | 46.5 |
| EC Erdölchemie | Solvent ICH | Isohexadecane | 43.8 |
| (supplier Bayer | | | |
| AG) | | | |
| DEA Mineralöl | Pionier 2076 | Mineral Oil | 43.7 |
| (Supplier Hansen | | | |
| & Rosenthal) | | | |
| Tudapetrol | | | |
| DEA Mineralöl | Pionier 6301 | Mineral Oil | 43.7 |
| (Supplier Hansen | | | |
| & Rosenthal) | | | |
| Tudapetrol | | | |
| Wacker | Wacker Silikonöl | Polydimethylsiloxane | 42.4 |
| | AK 35 | | |
| EC Erdölchemie | Isoeikosan | Isoeicosane | 41.9 |
| GmbH | | | |
| Wacker | Wacker Silikonöl | Polydimethylsiloxane | 40.9 |
| | AK 20 | | |
| Condea Chemie | Isofol 1212 | | 40.3 |
| | Carbonat | | |
| Gattefossé | Softcutol O | Ethoxydiglycol oleate | 40.5 |
| Creaderm | Lipodermanol OL | Decyl olivate | 40.3 |
| Henkel | Cetiol S | Dioctylcyclohexane | 39.0 |
| DEA Mineralöl | Pionier 2071 | Mineral oil | 38.3 |
| (supplier Hansen & | | | |
| Rosenthal) | | | |
| Tudapetrol | | | |
| WITCO BV | Hydrobrite 1000 | Paraffinum liquidum | 37.6 |
| | PO | | |
| Goldschmidt | Tegosoft HP | Isocetyl palmitate | 36.2 |
| Condea Chemie | Isofol Ester 1693 | | 33.5 |
| Condea Chemie | Isofol Ester 1260 | | 33.0 |
| Dow Corning | Dow Corning Fluid | Cyclopentasiloxane | 32.3 |
| | 245 | | |
| Unichema | Prisorine 2036 | Octyl isostearate | 31.6 |
| Henkel Cognis | Cetiol CC | Dicaprylyl carbonate | 31.7 |
| ALZO (ROVI) | Dermol 99 | Trimethylhexyl isononanoate | 31.1 |
| ALZO (ROVI) | Dermol 89 | 2-Ethylhexyl isononanoate | 31.0 |
| Henkel Cognis | Cetiol OE | Dicaprylyl ether | 30.9 |
| | Dihexylcarbonat | Dihexyl carbonate | 30.9 |
| Albemarle S.A. | Silkflo 366 NF | Polydecene | 30.1 |
| Unichema | Estol 1540 EHC | Octyl cocoate | 30.0 |

However, it is also advantageous to use mixtures of higher and lower polar lipids and the like. The oil phase can thus be advantageously chosen from the group of branched and unbranched hydrocarbons and hydrocarbon waxes, dialkyl ethers, the group of saturated or unsaturated, branched or unbranched alcohols, and the fatty acid triglycerides, namely the triglycerin esters of saturated and/or unsaturated, branched and/or unbranched alkanecarboxylic acids having a chain length of 8 to 24, in particular 12-18 C atoms. The fatty acid triglycerides can be advantageously chosen, e.g., from the group of synthetic, semi-synthetic and naturally occurring oils, for example, olive oil, sunflower oil, soybean oil, peanut oil, rapeseed oil, almond oil, palm oil, coconut oil, palm kernel oil and the like, provided the conditions required in the main claim are observed.

Fatty and/or wax components which are to be used advantageously according to the invention can be chosen from the group of vegetable waxes, animal waxes, mineral waxes and petrochemical waxes. Examples which are favorable according to the invention are candelilla wax, carnauba wax, japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microcrystalline waxes provided the conditions required in the main claim are observed.

Other advantageous fatty and/or wax components are chemically modified waxes and synthetic waxes, such as, for example, those obtainable under the trade names Syncrowax HRC (glyceryl tribehenate) and Syncrowax AW 1C (C₁₈₋₃₆-fatty acid) from CRODA GmbH, and montan ester waxes, Sasol waxes, hydrogenated jojoba waxes, synthetic or modified beeswaxes (e.g. dimethicone copolyol beeswax and/or C₃₀₋₅₀ alkyl beeswax), polyalkylene waxes, polyethylene glycol waxes, but also chemically modified fats, such as, for example, hydrogenated vegetable oils (for example, hydrogenated castor oil and/or hydrogenated coconut fatty glycerides), triglycerides, such as, for example, trihydroxystearin, fatty acids, fatty acid esters, and glycol esters, such as, for example, C₂₀₋₄₀-alkyl stearate, C20-40-alkylhydroxystearoyl stearate and/or glycol montanate. Also advantageous are certain organosilicon compounds, which have similar physical properties to the specified fatty and/or wax components, such as, for example, stearoxytrimethylsilane, provided the conditions required in the main claim are observed.

According to the invention, the fatty and/or wax components can be present either individually or as a mixture. Any desired mixtures of such oil and wax components can also be used advantageously for the purposes of the present invention.

The oil phase is advantageously chosen from the group of 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric triglyceride, dicaprylyl ether, provided the conditions required in the main claim are observed.

Particularly advantageous mixtures are those of octyldodecanol, caprylic/capric triglyceride, dicaprylyl ether, dicaprylyl carbonate, cocoglycerides, or mixtures of C₁₂₋₁₅-alkyl benzoate and 2-ethylhexyl isostearate, mixtures of C₁₂₋₁₅-alkyl benzoate and butylene glycol dicaprylate/dicaprate, and mixtures of C₁₂₋₁₅-alkyl benzoate, 2-ethylhexyl isostearate and isotridecyl isononanoate, provided the conditions required in the main claim are observed.

Of the hydrocarbons, paraffin oil, cycloparaffin, squalane, squalene, hydrogenated polyisobutene and polydecene are to be used advantageously for the purposes of the present invention, provided the conditions required in the main claim are observed.

Silicones

It may likewise be advantageous to choose some or all of the oil phase of the preparations according to the invention from the group of cyclic and/or linear silicones which are also referred to for the purposes of the present disclosure as “silicone oils.” Such silicones or silicone oils may be present as monomers which are generally characterized by structural elements as follows:

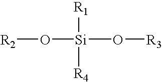

Silicone oils are high molecular weight synthetic polymeric compounds in which silicon atoms are joined in a catenated or reticular manner via oxygen atoms and the remaining valencies of the silicon are saturated by hydrocarbon radicals (in most cases methyl groups, more rarely ethyl, propyl, phenyl groups, etc.).

Linear silicones having two or more siloxyl units which are to be used advantageously according to the invention are generally characterized by structural elements as follows: 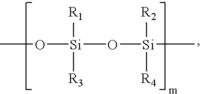
where the silicon atoms may be substituted by identical or different alkyl radicals and/or aryl radicals, which are represented here in general terms by the radicals R₁-R₄ (it should be said that the number of different radicals is not necessarily limited to 4). m may assume values from 2 to 200,000.

Systematically, the silicone oils are referred to as polyorganosiloxanes. The methyl-substituted polyorganosiloxanes, which represent the most important compounds of this group in terms of amount and are characterized by the following structural formula 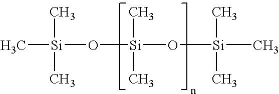
are also referred to as polydimethylsiloxane or dimethicone (INCI). Dimethicones come in various chain lengths and with various molecular weights. Dimethicones of different chain length and phenyltrimethicones are particularly advantageous linear silicone oils for the purposes of the present invention.

For the purposes of the present invention, particularly advantageous polyorganosiloxanes are, for example, dimethylpolysiloxanes [poly(dimethylsiloxane)], which are available, for example, under the trade name ABIL 10 to 10,000 from Th. Goldschmidt. Also advantageous are phenylmethylpolysiloxanes (INCI: phenyl dimethicone, phenyl trimethicone), cyclic silicones (octamethylcyclotetrasiloxane and decamethylcyclopentasiloxane), which are also referred to in accordance with INCI as cyclomethicones, amino-modified silicones (INCI: amodimethicones) and silicone waxes, e.g., polysiloxane-polyalkylene copolymers (INCI: stearyl dimethicone and cetyl dimethicone) and dialkoxydimethylpolysiloxane (stearoxy dimethicone and behenoxy stearyl dimethicone), which are available as various Abil-wax grades from Th. Goldschmidt.

Furthermore, the silicone oils listed below are particularly advantageous for the purposes of the present invention:

| | | | Polarity |
|---|---|---|---|
| Manufacturer | Trade Name | INCI Name | [mN/m] |
| Wacker | Wacker Silikonöl AK | Polydimethylsiloxane | 26.9 |
| | 100 | | |
| Wacker | Wacker Silikonöl | Polydimethylsiloxane | 46.5 |
| | AK 50 | | |
| Wacker | Wacker Silikonöl AK | Polydimethylsiloxane | 42.4 |
| | 35 | | |
| Wacker | Wacker Silikonöl AK | Polydimethylsiloxane | 40.9 |
| | 20 | | |
| Dow Corning | Dow Corning Fluid | Cyclopentasiloxane | 32.3 |
| | 245 | | |
| Dow Corning | Dow Corning Fluid | Cyclomethicone | 28.5 |
| | 345 | | |

Cyclic silicones to be used advantageously according to the invention are generally characterized by structural elements, as follows: 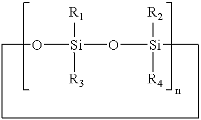
whereby the silicon atoms can be substituted with the same or different alkyl residues and/or aryl residues, which are shown here in general terms by the residues R₁-R₄ (in other words, the number of different residues is not necessarily restricted to 4). For this, n can assume values from 3/2 to 20. Broken values for n allow for the fact that non-linear numbers of siloxyl groups can be present in the cycle.

Particularly advantageous cyclic silicone oils to be used advantageously for the purposes of the present invention are cyclomethicones, in particular cyclomethicone D5 and/or cyclomethicone D6.

Advantageous silicone oils or silicone waxes for the purposes of the present invention are cyclic and/or linear silicone oils and silicone waxes.

It is particularly advantageous for the purposes of the present invention to choose the ratio of lipids to silicone oils to be approx. 1:1 (generally x:y).

Phenylmethicone is advantageously chosen as silicon oil. Also other silicon oils, e.g., dimethicone, phenyldimethicone, cyclomethicone (octamethylcyclotetrasiloxane), e.g., hexamethylcyclotrisiloxane, polydimethylsiloxane, poly(methylphenylsiloxane), cetyldimethicone, behenoxydimethicone, can be advantageously used for the purposes of the present invention.

Furthermore, mixtures of cyclomethicone and isotridecylisononanoate, and those of cyclomethicone and 2-ethylhexyl isostearate are also advantageous.

It is, however, also advantageous to choose silicone oils of similar constitution to the above-described compounds whose organic side chains are derivatized, for example polyethoxylated or polypropoxylated. These include, for example, polysiloxane-polyalkyl-polyether copolymers, such as cetyl-dimethicone copolyol, (cetyl-dimethicone copolyol (and) polyglyceryl-4 isostearate (and) hexyl laurate.

The preparations according to the invention can according to the invention advantageously contain one or more emollients or humectants. Advantageous emollients or humectants (so-called moisturizers) for the purposes of the present invention are, e.g., glycerin, lactic acid and/or lactates, in particular sodium lactate, butylene glycol, propylene glycol, biosaccaride gum-1, glycine soy, ethylhexyloxyglycerin, pyrrrolidone carboxylic acid and urea. Furthermore, it is particularly advantageous to use polymeric moisturizers from the group of polysaccharides which are water-soluble and/or water-swellable and/or gellable using water. Particularly advantageous are, for example, hyaluronic acid, chitosan and/or a fucose-rich polysaccharide which is filed in the Chemical Abstracts under the registry number 178463-23-5 and is available, for example, under the name Fucogel® 1000 from SOLABIA S. A.

According to the invention, the preparations according to the invention can also advantageously contain vitamins and vitamin derivatives and antioxidants. These include, inter alia, the vitamins A, B₁₋₆, B₁₂, C, D, E, F, H, K, and PP, as well as their derivatives. According to the invention, they may be advantageously contained in a concentration of from 0.001 to 10 wt. %, preferably 0.05 to 7 wt. %, more preferably 0.5 to 5 wt. %, each based on the total weight of the preparation.

Retinyl palmitate, ascorbyl glucoside, tocopheryl acetate, tocopheryl palmitate, niacinamide, and panthenol are used as preferred vitamin derivates in accordance with the invention.

Further advantageous active ingredients for the purposes of the present invention are natural active ingredients and derivatives thereof, such as, for example, alpha-lipoic acid, phytoene, D-biotin, coenzyme Q10, alpha-glucosylrutin, carnitine, carnosine, natural and/or synthetic isoflavonoids, creatine, creatinine, lignans, taurine, and/or β-alanine.

However, other pharmaceutically or dermatologically effective substances, such as, for example, substances which calm and care for the skin, can also be incorporated into the preparations according to the invention. These include, for example, panthenol, allantoin, tannin, and plant active ingredients, such as azulene and bisabolol, glycyrrhizin, hamamelin and plant extracts, such as camomile, aloe vera, hamamelis, liquorice.

The amount of these active ingredients (one or more compounds) present in the preparations is preferably 0.001 to 30% by weight, particularly preferably 0.05-20% by weight, in particular 1-10% by weight, based on the total weight of the preparation.

According to the invention, the compositions may contain besides the foregoing substances, additional substances as are customary in cosmetics, for example, perfume, dyes, antimicrobial agents, refatting agents, complexing and sequestering agents, pearlescent agents, plant extracts, selftanners (for example, DHA), depigmentors, antidandruff ingredients, vitamins, additional active ingredients, complexes of gamma-oryzanol and calcium salts, niacinamide and its derivatives, panthenol and its derivatives, subtilisin, minerals, preservatives, bactericides, pigments which have a coloring effect, thickeners, softeners, moisturizers, and/or humectants, or other customary constituents of a cosmetic or dermatological formulation, such as alcohols, polyols, polymers, foam stabilizers, electrolytes, organic solvents or silicone derivatives. The pH value of the preparations according to the invention is adjusted in a manner customary to one skilled in the art with the corresponding acids (lactic acid, citric acid, phosphoric acid, etc.), and bases (for example, NaOH).

The following examples are to illustrate the compositions according to the invention with no intention of restricting the invention to these examples. The numerical values in the examples mean % by weight, based on the total weight of the respective preparations.

### EXAMPLES

| | Example No. | | | | | |
|---|---|---|---|---|---|---|
| | 1 | 2 | 3 | 4 | 5 | 6 |
| MIPA laureth sulfate | 20.0 | 25.0 | 15.0 | — | — | — |
| Sodium myreth sulfate | — | — | — | 8.0 | — | — |
| Sodium laureth sulfate | — | — | — | — | 5.0 | 3.0 |
| Sodium cocoamphoacetate | — | 0.5 | — | — | 2.0 | — |
| Lauryl glucoside | — | 0.5 | 0.5 | — | 1.0 | 1.0 |
| Mineral oil | 30.0 | 20.0 | 20.0 | 30.0 | — | — |
| C12-15 Alkyl benzoate | 20.0 | 10.0 | 15.0 | — | 5.0 | 3.0 |
| Octyldodecanol | 13.7 | — | — | 10.0 | 5.0 | 3.0 |
| Soybean oil | 15.0 | 35.0 | — | 15.0 | 25.0 | 2.0 |
| Cetyl palmitate | — | 5.0 | — | — | 2.0 | — |
| Hydroxypropyl | — | — | — | — | 1.0 | 0.5 |
| methylcellulose | | | | | | |
| Glycerin | — | — | 2.0 | 5.0 | 3.0 | 1.0 |
| BHT | 1.0 | 2.0 | — | 0.2 | — | — |
| NA₃HEDTA | — | — | 1.0 | — | 1.0 | 1.0 |
| Sodium hydroxide | — | 0.25 | 0.5 | — | — | — |
| Citric acid | — | 0.25 | 0.5 | — | — | — |
| Colorant, perfume | 0.3 | 0.5 | — | — | 0.5 | 0.5 |
| Water | — | 1.0 | Ad | Ad | Ad | Ad |
| | | | 100 | 100 | 100 | 100 |

1-10. (canceled)

11. A cleansing article, wherein the cleansing article comprises a textile that is impregnated with a preparation comprising:
(a) from 1% to 30% by weight of one or more anionic surfactants; and
(b) at least 8% by weight of one or more lipids which are liquid or viscous at room temperature,
each based on a total weight of the preparation.

12. The article of claim 11, wherein the preparation comprises at least 20% by weight of (b).

13. The article of claim 12, wherein the preparation comprises from 2% to 25% by weight of (a).

14. The article of claim 11, wherein (b) comprises one or more of mineral oil, C₁₂₋₁₅ alkyl benzoate, octyldodecanol, soybean oil and cetyl palmitate.

15. The article of claim 11, wherein (a) comprises one or more of sodium myreth sulfate, sodium laureth sulfate, monoisopropanolamine laureth sulfate, sodium acyl glutamate, sodium lauroyl sarcosinate, sodium methyl cocoyl taurate and dioctyl sodium sulfosuccinate.

16. The article of claim 11, wherein the textile comprises a nonwoven fabric.

17. The article of claim 11, wherein the textile comprises a wipe.

18. The article of claim 11, wherein the textile comprises from 0% to 30% by weight of cotton fibers, from 10% to 80% by weight of viscose fibers and from 20% to 90% by weight of polyester, each based on a total weight of the textile.

19. The article of claim 11, wherein the textile has a degree of impregnation of from 100% to 1000%.

20. The article of claim 11, wherein the article comprises a textile that is impregnated with a preparation comprising:
(a) from 2% to 25% by weight of one or more anionic surfactants which comprise one or more of sodium myreth sulfate, sodium laureth sulfate, monoisopropanolamine laureth sulfate, sodium acyl glutamate, sodium lauroyl sarcosinate, sodium methyl cocoyl taurate and dioctyl sodium sulfosuccinate; and
(b) at least 20% by weight of one or more lipids which are liquid or viscous at room temperature and comprise one or more of mineral oil, C₁₂₋₁₅ alkyl benzoate, octyldodecanol, soybean oil and cetyl palmitate,
each based on a total weight of the preparation.

21. The article of claim 20, wherein the textile comprises a nonwoven fabric.

22. The article of claim 21, wherein the textile comprises a wipe.

23. The article of claim 22, wherein the textile comprises from 0% to 30% by weight of cotton fibers, from 10% to 80% by weight of viscose fibers and from 20% to 90% by weight of polyester, each based on a total weight of the textile.

24. A process for producing a cleansing article, wherein the process comprises an impregnation of a textile with a preparation comprising
(a) from 1% to 30% by weight of one or more anionic surfactants; and
(b) at least 8% by weight of one or more lipids which are liquid or viscous at room temperature,
each based on a total weight of the preparation.

25. The process of claim 24, wherein the impregnation comprises at least one of immersion of the textile in a dip bath, spraying the textile with the preparation and wiping the textile on doctor blades, doctor bars, or doctor nozzles which discharge the preparation.

26. The process of claim 25, wherein the preparation comprises from 2% to 25% by weight of (a) and at least 20% by weight of (b).

27. The process of claim 26, wherein (a) comprises one or more of sodium myreth sulfate, sodium laureth sulfate, monoisopropanolamine laureth sulfate, sodium acyl glutamate, sodium lauroyl sarcosinate, sodium methyl cocoyl taurate and dioctyl sodium sulfosuccinate and (b) comprises one or more of mineral oil, C₁₂₋₁₅ alkyl benzoate, octyldodecanol, soybean oil and cetyl palmitate.

28. The process of claim 25, wherein the textile comprises a nonwoven fabric.

29. The process of claim 28, wherein the textile comprises from 0% to 30% by weight of cotton fibers, from 10% to 80% by weight of viscose fibers and from 20% to 90% by weight of polyester, each based on a total weight of the textile.

30. The process of claim 29, wherein the textile has a degree of impregnation of from 100% to 1000%.

31. A method of producing cleansing wipes, wherein the method comprises impregnating a textile with a preparation which comprises
(a) from 1% to 30% by weight of one or more anionic surfactants; and
(b) at least 8% by weight of one or more lipids which are liquid or viscous at room temperature,
each based on a total weight of the preparation,
and wherein the wipes foam upon application to the skin and refat the skin with lipids.
